(19) United States
(12) Patent Application Publication
(10) Pub. No.: US 2016/0303078 A1
(43) Pub. Date: Oct. 20, 2016

(54) METHODS OF MODULATING VARIOUS BIOMARKERS WITH CURAXINS

(71) Applicants: Ivan BESPALOV, Buffalo, NY (US); Incuron, LLC, Buffalo, NY (US)

(72) Inventor: Ivan Bespalov, Buffalo, NY (US)

(73) Assignee: Incuron, LLC, Buffalo, NY (US)

(21) Appl. No.: 15/103,036

(22) PCT Filed: Dec. 10, 2014
(86) PCT No.: PCT/US14/69552

§ 371 Date: Jun. 9, 2016

Related U.S. Application Data
(60) Provisional application No. 61/914,215, filed on Dec. 10, 2013.

(51) Int. Cl. A61K 31/403; G01N 33/574

(52) U.S. Cl. CPC A61K 31/403; G01N 33/57484; G01N 2800/52

(57) ABSTRACT

The present invention relates to improved methods for diagnosing and/or treating cancer with a curaxin, including curaxin 137.

### CROSS-REFERENCE TO RELATED APPLICATIONS

This application claims the benefit of, and priority to, U.S. Provisional Patent Application No. 61/914,215, filed Dec. 10, 2013, the entire contents of which are incorporated herein by reference.

### FIELD OF THE INVENTION

The present invention relates to methods that are useful in treating or diagnosing cancer, including for example, with curaxins.

### BACKGROUND

Cancer is a leading cause of death in most countries and the cause of billions of dollars in healthcare expense around the world. Many cancers are caused, at least in part, by genetic abnormalities that result in either the overexpression of cancer causing genes, including oncogenes or loss of function mutations in protective genes, including tumor suppressor genes. An example of the latter is p53 (also known as protein 53, tumor protein 53, tumor antigen p53, etc.)—a 53 kD nuclear phosphoprotein that controls cell proliferation. Mutations to the p53 gene and allele loss on chromosome 17p, where this gene is located, are among the most frequent alterations identified in human malignancies. Wild-type p53 has been shown to be involved in control of the cell cycle, transcriptional regulation, DNA replication, and induction of apoptosis. As such, p53 has been described as “the guardian of the genome” because of its role in conserving stability by preventing genome mutation. Accordingly, cancer treatments and diagnostics would benefit from agents that can modulate p53 and restore it genomic protection effect.

Further, personalized medicine approaches assist in tailoring patient care to maximize anti-cancer efficacy and minimize deleterious side effects. Accordingly, the field requires biomarker assays that can serve as diagnostic tools to predict and evaluate a drugs utility in a patient.

Therefore, there remains a need for methods that are useful for treating or diagnosing cancer and related diseases, including on a personalized basis.

### BRIEF DESCRIPTION OF THE INVENTION

Accordingly, the present invention relates to improved methods for treating cancer, including, for example, cancer characterized by reduced levels of p53 and/or p53 activity, with agents that can modulate p53 and restore its genomic protection effect. The invention also provides methods for predicting patient response to various agents, including curaxins (e.g. Curaxin 137) by monitoring one or more biomarkers (e.g. p53 and/or FACT and/or a NF-kB-responsive marker (e.g. CXCL10) and/or CXCL10/IP-10, CXCL9/MIG, IFIT3 and/or NF-kB) and directing patient care accordingly.

In one aspect, the present invention provides a method for treating cancer, comprising administering an effective amount of a curaxin compound, as described in PCT/US2009/059558 (international Patent Publication No. WO 2010/042445), the entire disclosure of which is incorporated herein by reference, and including, but not limited to a compound of Formula I to a subject in need thereof:

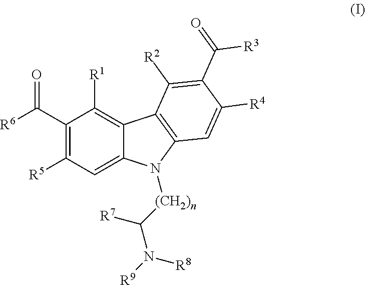

wherein each of R¹-R⁹, are independently H, hydroxyl or alkyl; n is 0, 1, 2, 3, 4, or 5; or a pharmaceutically acceptable salt or hydrate thereof; and the cancer is characterized by having reduced levels of p53 and/or p53 activity relative to a non-cancerous state.

In another aspect, the present invention pertains to a method for increasing levels of p53 and/or p53 activity, comprising administering an effective amount of a curaxin compound, including but not limited to a compound of Formula I to a subject in need thereof, wherein the levels of p53 and/or p53 activity in blood are increased greater than at least 5-fold relative to an untreated state.

In another aspect, the present invention provides a method for identifying a subject with a tumor that is likely to respond to treatment with a curaxin compound, including but not limited to a compound of Formula I comprising evaluating the tumor and/or a blood sample, comprising measuring a presence, absence, level, or activity of p53 and/or a NF-kB-responsive marker (e.g. CXCL10) and/or NF-kB, wherein a reduced level or activity of p53 and/or a NF-kB-responsive marker (e.g. CXCL10) and/or NF-kB kB relative to a non-tumor state indicates that the subject is likely respond to treatment with a curaxin compound, including but not limited to a compound of Formula I, and optionally directs administering an effective amount of the curaxin compound, including but not limited to a compound of Formula I, to a subject that is likely to respond to curaxin compound, including but not limited to a compound of Formula I.

In another aspect, the present invention provides a method for identifying a subject with a tumor that is likely to respond to treatment with a curaxin compound, including but not limited to a compound of Formula I comprising evaluating the tumor and/or a blood sample, comprising measuring a ratio of chromatin-bound FACT to soluble FACT, wherein an increased ratio of chromatin-bound FACT to soluble FACT relative to a non-tumor state indicates that the subject is likely respond to treatment with a curaxin compound, including but not limited to a compound of Formula I and optionally directs administering an effective amount of the curaxin compound, including but not limited to a compound of Formula I to a subject that is likely to respond to the curaxin compound, including but not limited to a compound of Formula I.

In yet another aspect, the present invention provides a method for a method for identifying a subject with a tumor that is likely to respond to treatment with an agent that targets or effects p53, comprising administering an effective amount of a curaxin compound, including but not limited to a compound of Formula I and evaluating the tumor and/or a blood sample, comprising measuring a presence, absence, level or activity of p53, wherein an increased level or activity of p53 relative to an untreated state indicates that the subject is likely respond to an agent that targets or effects p53.

In one aspect, the present invention provides a method for treating cancer, comprising administering an effective amount of a curaxin compound including, but not limited to a compound of Formula I to a subject in need thereof, wherein the subject's level or activity of p53 is elevated to greater than 100 pg/ml after treatment. In some embodiments, the method further comprises measuring the subject's level or activity of p53. In some embodiments, the cancer is characterized by having reduced levels of p53 and/or p53 activity relative to a non-cancerous state.

In various embodiments, the compound of Formula I is:

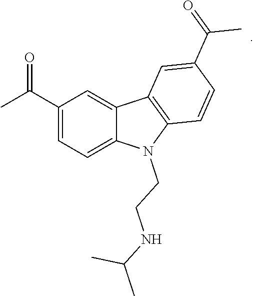 [SMILES: CC(=O)c1ccc2c(c1)c1cc(C(C)=O)ccc1n2CCNC(C)C]

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF THE DRAWINGS

FIG. 1 shows, without wishing to be bound by theory, a possible mechanism of action for the curaxins.

FIG. 2 shows the effect of Curaxin137 on the ratio between bound FACT and free FACT as assayed by ELISA.

FIG. 3 shows the effect of Curaxin137 on expression of p53 in peripheral blood mononuclear cells (PBMC) in a human patient.

FIG. 4 shows the effect of curaxins on NF-kB suppression. CXCL10 is encoded by an NF-kB-regulated gene.

FIG. 5 shows SSRP1 concentration changes (normalized for protein, Y axis) in PBMC extracts within 24 hr time prriod after administration of CBL0137 in a human patient. Soluble SSRP1 is extracted in 2× PBS fraction; 4× PBS fractions contain mobilized SSRP1. Total is the sum of SSRP1 concentrations in all extracts.

FIG. 6 shows levels of p53 in PBMC samples collected within 72 hours after administration of CBL0137. A representative human data set is shown.

FIG. 7 shows CXCL10/IP-10 cohort-average percentage changes relative to predose within 48 hour time period after CBL0137 administration.

FIG. 8 shows relative IFIT3 levels as percentages of pre-treatment baseline measurements. One human patient data is shown. The X axis shows time in hours or days (“c” is cycle, i.e. a 28 day period of time consisting of 2 weeks of once daily oral treatment with CBL0137 followed by 2 weeks of rest and “d” is days within the cycle). IFIT3 in plasma gradually increased, starting with Cycle 1 Day 7 and reaching a 80% higher level compared to predose by the end of treatment (Cycle 2 Day 28).

### DETAILED DESCRIPTION OF THE INVENTION

The present invention is based, in part, on the discovery that curaxins, including Curaxin 137 (a/k/a CBL0137, CBLB137) can cause an unexpectedly marked increase in levels of p53 and have an anti-cancer effect in cancers that are characterized by having low levels of p53. The present invention is based, in part, on the discovery that measurement of certain biomarkers (e.g. p53 and/or a NF-kB-responsive marker (e.g. CXCL10) and/or NF-kB and/or FACT) correlate with effect of the curaxin, including Curaxin 137, and can be useful in directing patient care. The present invention is based, in part, on the discovery that certain biomarkers in the blood can be useful in evaluating curaxin drug effects on solid tumors.

In one aspect, the present invention provides a method for treating cancer, comprising administering an effective amount of a curaxin compound, including but not limited to a compound of Formula I, to a subject in need thereof:

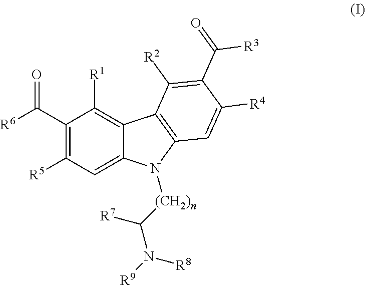

wherein each of R¹-R⁹ are independently H, hydroxyl or alkyl; n is 0, 1, 2, 3, 4, or 5; or a pharmaceutically acceptable salt or hydrate thereof; and the cancer is characterized by having reduced levels or activity of p53 relative to a non-cancerous state.

In various embodiments, the compound of Formula I is curaxin 137:

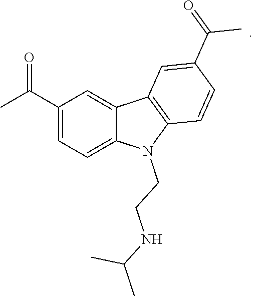 [SMILES: CC(=O)c1ccc2c(c1)c1cc(C(C)=O)ccc1n2CCNC(C)C]

In various embodiments, the curaxin compound, including but not limited to a compound of Formula I, is administered in combination with an additional therapy, including, by way of non-limiting example, an agent that targets or effects p53. In various embodiments, the curaxin compound, including but not limited to a compound of Formula I, is administered as an adjuvant therapy after resection. In various embodiments, the curaxin compound, including but not limited to a compound of Formula I, is sole adjuvant therapy. In various embodiments, the curaxin compound, including but not limited to a compound of Formula I, is administered as an adjuvant therapy prior to resection. In various embodiments, the subject is a human. In various embodiments, the cancer is resistant and/or non-responsive to an agent that targets or effects p53 based on a chemosensitivity test or surrogate biomarker.

In another aspect, the present invention pertains to a method for increasing levels or activity of p53, comprising administering an effective amount of a curaxin compound, including but not limited to a compound of Formula I, to a subject in need thereof, wherein the levels or activity of p53 in blood are increased greater than at least 5-fold relative to an untreated state.

In various embodiments, the compound of Formula I is curaxin 137.

In various embodiments, the levels or activity of p53 are increased by about 5-fold, or about 7.5-fold, or about 10 fold, or about 12.5 fold, or about 15 fold. In various embodiments, the levels or activity p53 are restored to non-cancerous levels or activities in the subject. In various embodiments, the p53 is wild type p53. In various embodiments, the levels or activity of wild type p53 are increased by about 5-fold, or about 7.5-fold, or about 10 fold, or about 12.5 fold, or about 15 fold. In various embodiments, the levels or activity of wild type p53 are restored to non-cancerous levels or activities in the subject.

In another aspect, the present invention provides a method for identifying a subject with a tumor that is likely to respond to treatment with a curaxin compound, including but not limited to a compound of Formula I, comprising evaluating the tumor and/or a blood sample, comprising measuring a presence, absence, level, or activity of one or more of p53 and/or a NF-kB-responsive marker (e.g. CXCL10) and/or CXCL10/IP-10. CXCL9/MIG, IFIT3 and/or NF-kB, wherein a reduced level or activity of p53 and/or NF-kB-responsive marker (e.g. CXCL10) and/or CXCL10/IP-10, CXCL9/MIG, IFIT3 and/or NF-kB relative to a non-tumor state indicates that the subject is likely respond to treatment with a curaxin compound, including but not limited to a compound of Formula I, and optionally directs administering an effective amount of the curaxin compound, including but not limited to a compound of Formula I, to a subject that is likely to respond to the curaxin compound, including but not limited to a compound of Formula I.

In another aspect, the present invention provides a method for identifying a subject with a tumor that is likely to respond to treatment with a curaxin compound, including but not limited to a compound of Formula I, comprising evaluating the tumor and/or a blood sample, comprising measuring a ratio of chromatin-bound FACT to soluble FACT, wherein an increased ratio of chromatin-bound FACT to soluble FACT relative to a non-tumor state indicates that the subject is likely respond to treatment with curaxin compound, including but not limited to a compound of Formula I, and optionally directs administering an effective amount of the curaxin compound, including but not limited to a compound of Formula I, to a subject that is likely to respond to the curaxin compound, including but not limited to a compound of Formula I.

In various embodiments, the compound of Formula I is curaxin 137.

In various embodiments, the measurement comprises evaluating a presence, absence, or level of a protein. In some embodiments, the measurement comprises contacting a specimen of the tumor or cells cultured from the tumor with an agent that specifically binds one or more of p53, CXCL10, NF-kB or FACT, including, for example, an antibody. In some embodiments, the measurement of one or more of p53, CXCL10, NF-kB or FACT comprises one or more of immunohistochemical staining, western blotting, in cell western, immunofluorescent staining. ELISA, and fluorescent activating cell sorting (FACS). In some embodiments, the tumor specimen is a biopsy selected from a frozen tumor tissue specimen, cultured cells, circulating tumor cells, and a formalin-fixed paraffin-embedded tumor tissue specimen.

In yet another aspect, the present invention provides a method for a method for identifying a subject with a tumor that is likely to respond to treatment with an agent that targets or effects p53, comprising administering an effective amount of a curaxin compound, including but not limited to a compound of Formula I, and evaluating the tumor and/or a blood sample, comprising measuring a presence, absence, level, or activity of p53, wherein an increased level or activity of p53 relative to an untreated state indicates that the subject is likely respond to an agent that targets or effects p53.

In various embodiments, the compound of Formula I is curaxin 137.

In various embodiments, the measurement comprises evaluating a presence, absence, or level of a protein. In some embodiments, the measurement comprises contacting a specimen of the tumor or cells cultured from the tumor with an agent that specifically binds p53, including, for example, an antibody. In some embodiments, the measurement of p53 comprises one or more of immunohistochemical staining, western blotting, in cell western, immunofluorescent staining, ELISA, and fluorescent activating cell sorting (FACS). In some embodiments, the tumor specimen is a biopsy selected from a frozen tumor tissue specimen, cultured cells, circulating tumor cells, and a formalin-fixed paraffin-embedded tumor tissue specimen.

The following definitions are used in connection with the invention disclosed herein. Unless defined otherwise, all technical and scientific terms used herein have the same meaning as commonly understood to one of skill in the art to which this invention belongs.

As used herein, “a,” “an,” or “the” can mean one or more than one. Further, the term “about” when used in connection with a referenced numeric indication means the referenced numeric indication plus or minus up to 10% of that referenced numeric indication. For example, the language “about 50” covers the range of 45 to 55.

The term “administer”, “administering”, or “administration” as used in this disclosure refers to either directly administering a compound or pharmaceutically acceptable salt of the compound or a composition to a subject, or administering a prodrug conjugate or analog of the compound or pharmaceutically acceptable salt of the compound or composition to the subject, which can form an equivalent amount of active compound within the subject's body.

The term “alkyl,” as used herein unless otherwise defined, refers to a straight or branched saturated group derived from the removal of a hydrogen atom from an alkane. Representative straight chain alkyl groups include, but are not limited to, -methyl, -ethyl, -n-propyl, -n-butyl, -n-pentyl, and n-hexyl. Representative branched alkyl groups include, but are not limited to, isopropyl, -sec-butyl, -isobutyl, -tert-butyl, -isopentyl, -neopentyl, 1-methylbutyl, 2-methylbutyl, 3-methylbutyl, 1,1-dimethylpropyl and 1,2-dimethylpropyl.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

The term “disorder” is used in this disclosure to mean, and is used interchangeably with, the terms disease, condition, or illness, unless otherwise indicated.

An “effective amount,” when used in connection with a compound described herein is an amount that is effective for providing a measurable treatment, prevention, or reduction in the pathogenesis of a cancer or related disease.

The term “hydroxyl” means —OH.

A “subject” is a mammal, e.g., a human (e.g. a female or a male human), mouse, rat, guinea pig, dog, cat, horse, cow, pig, or non-human primate, such as a monkey, chimpanzee, baboon or rhesus, and the terms “subject” and “patient” are used interchangeably herein.

The term “treating”, with regard to a subject, refers to improving at least one symptom of the subject's disorder. Treating can be curing, improving, or at least partially ameliorating the disorder, including cancer or a related disease. Treatment can be assessed using various endpoints, including overall survival, progression-free interval, disease-free interval, or pathological complete response.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

p53 is crucial in multicellular organisms, where it regulates the cell cycle and, thus, functions as a tumor suppressor that is involved in preventing cancer. As such, p53 has been described as the guardian of the genome” because of its role in conserving stability by preventing genome mutation.

In various embodiments, the present invention pertains to p53. p53 has many mechanisms of anticancer function, and plays a role in, by way of non-limiting example, apoptosis, genomic stability, and inhibition of angiogenesis. In its anti-cancer role, p53 works through several mechanisms. Without wishing to be bound by theory, illustrative mechanisms include: activation of DNA repair proteins when DNA has sustained damage, arresting of cell growth by holding the cell cycle at the G2₁/S regulation point on DNA damage recognition; and initiation of apoptosis if DNA damage proves to be irreparable. In a normal cell p53 is inactivated by its negative regulator, mdm2. Upon stress (including, but not limited to, DNA damage (induced by, for example, UV, IR, or chemical agents such as hydrogen peroxide), oxidative stress, osmotic shock, ribonucleotide depletion, and deregulated oncogene expression) various pathways will lead to the dissociation of the p53 and mdm2 complex. Once activated, p53 will induce a cell cycle arrest to allow either repair and survival of the cell or apoptosis to discard the damaged cell. Activated p53 binds DNA and activates expression of several genes including, but not limited to, microRNA miR-34a, WAF1/CIP1 encoding for p21 and hundreds of other down-stream genes. p21 (WAF1) binds to the G1-S/CD (CDK2) and S/CDK complexes (molecules important for the G1/S transition in the cell cycle) inhibiting their activity.

If the TP53 gene is damaged, tumor suppression is severely reduced. Subjects with only one functional copy of the TP53 gene will most likely develop tumors in early adulthood (Li-Fraumeni syndrome). The TP53 gene can also be damaged in cells by mutagens (chemicals, radiation, or viruses), increasing the likelihood that the cell will begin decontrolled division. More than 50 percent of human tumors contain a mutation or deletion of theTP53 gene, p53 is linked to many, but not all, cancer types. For example, p53 may not play a role in, for example, colorectal cancer and endometrial cancer risk.

In some embodiments, the present invention provides for the increasing the amount of p53 in the treatment of cancer or as a diagnostic tool. In some embodiments, the present invention provides for restoring endogenous p53 levels and activity. In some embodiments, the present invention pertains to methods of modulating p53, including increasing the expression of p53. In some embodiments, the invention provides restoration of p53's tumor suppressing activity, including causing a functional restoration in subjects without mutations in p53 itself but with a partially abrogated p53 pathway (e.g. causing restoration in subjects characterized by having an overexpression of negative regulators of p53, such as, for example, MDM2 and MDM4, and/or deletion or epigenetic inactivation of the positive regulators of p53, such as, for example, ARF). In some embodiments, the invention provides restoration of p53's tumor suppressing activity, including causing a restoration of p53 levels or activity in which p53 is inactivated via p53 mutation (e.g. in the core DNA-binding domain).

In some embodiments, the compounds described herein increase the amount of p53 in a subject such that it is greater than in a diseased (e.g. cancerous) state. In some embodiments, the compounds described herein increase the amount of p53 in a subject such that it is greater than in an untreated state. In some embodiments the increase is at least 3-, or 4-, or 5-, or 6-, or 7-, or 7.5-, or 8-, or 9-, or 10-, or 11-, or 12-, or 12.5, or 13-, or 14-, or 15-fold. In some embodiments the increase is about 3-, or about 4-, or about 5-, or about 6-, or about 7-, or about 7.5-, or about 8-, or about 9-, or about 10-, or about 11-, or about 12-, or about 12.5, or about 13-, or about 14-, or about 15-fold.

In some embodiments, p53 measurement is useful as a companion diagnostic assay for curaxin, including Curaxin 137 response. in some embodiments, the amount of p53 is useful to predict a subject's response to a curaxin, including Curaxin 137. In some embodiments, an increased level or activity of p53 in a subject's sample upon curaxin treatment directs the administering of a curaxin, including Curaxin 137 to the subject. Conversely, in some embodiments, a reduced or unchanged amount of p53 in a subject's sample upon curaxin treatment directs away from the administering of a curaxin, including Curaxin 137 to the subject.

In some embodiments, the present invention provides for a diagnostic for curaxin response in cancers comprising measuring level or activity of p53 in blood. In some embodiments, the present invention provides for a diagnostic for curaxin response in solid tumors comprising measuring level or activity of p53 in blood. In some embodiments, the measurement comprises an ELISA.

In various embodiments, the diagnostic techniques described herein comprise a measurement of p53 levels or activity in a sample (e.g. blood). In some embodiments, the diagnostic techniques described herein comprise a measurement of baseline or normal levels. In some embodiments, this measurement is before or at the administering a curaxin to a subject. In some embodiments, further measurements occur at about 10, or about 20, or about 30, or about 40, or about 50 hours after administering a curaxin to a subject. In some embodiments, further measurements occur at about a day, or two days after administering a curaxin to a subject.

In various embodiments, the present methods allow for dose determination and regulation via measurement of p53 levels or activity in a sample (e.g. blood). For example, a medical practitioner can optimize dose by measuring the levels or activity in a sample (e.g. blood) and altering dose to correlate with a level of p53 that is reflective of curaxin activity.

In one aspect, the present invention provides a method for treating cancer, comprising administering an effective amount of a curaxin compound including, but not limited to a compound of Formula I to a subject in need thereof, wherein the subject's level or activity of p53 is elevated to greater than 100 pg/ml after treatment. In some embodiments, the method further comprises measuring the subject's level or activity of p53. In some embodiments, the measurement occurs at about 10, or about 20, or about 30, or about 40, or about 50 hours after administering a curaxin to a subject. In some embodiments, further measurements occur at about a day, or two days after administering a curaxin to a subject. In some embodiments, the subject's level or activity of p53 is elevated to about 110 pg/ml, or about 120 pg/ml, or about 130 pg/ml, or about 140 pg/ml, or about 150 pg/ml, or about 160 pg/ml, or about 170 pg/ml, or about 180 pg/ml, or about 190 pg/ml, or about 200 pg/ml, or about 210 pg/ml, or about 220 pg/ml after treatment. In various embodiments, the measurement is made with blood samples.

in various embodiments, the present invention pertains to the FAcilitates Chromatin Transcription (FACT) complex, a heterodimer of two subunits: an 80 kDa subunit and a 140 kDa subunit. These subunits are Structure Specific Recognition Protein 1 (SSRP1) and Suppressor of Ty (SPT16 or SUPT16H). As used herein, FACT refers to the heterodirner SSRP1 and SPT16, or the individual SSRP1 and SPT16 subunits. Without wishing to be bound by theory, FACT is involved in chromatin remodeling through modulating of nucleosome stability. FACT may be involved in many processes involving chromatin, such as, for example, transcription, replication, recombination. DNA damage, and repair. FACT interacts specifically with histones H2A/H2B to effect nucleosome disassembly and transcription elongation. Without wishing to be bound by theory, curaxins (e.g, Curaxin-137), small molecules which have broad anti-cancer activity in different models of cancer cause functional inactivation of FACT (See Gasparian, et al. Sci. Trans. Med. 3:95ra74 (2011), the contents of which are hereby incorporated by reference in their entirety).

The protein encoded by the gene of structure specific recognition protein 1 (SSRP1)(mRNA in humans: NM_003146.2, the sequence is hereby incorporated by reference in its entirety, mRNA in mouse: NM_001136081.1, the sequence is hereby incorporated by reference in its entirety) is a subunit of a heterodimer that, along with SPT16, forms FACT, SSRP1 is the 80 kDa subunit. FACT and cisplatin-damaged DNA may be crucial to the anticancer mechanism of cisplatin. SSRP1 encoded protein (in humans: NP_003137.1, the sequence is hereby incorporated by reference in its entirety, in mouse: NP_001129553.1, the sequence is hereby incorporated by reference in its entirety) contains a high mobility group box which, without wishing to be bound by theory, may constitutes the structure recognition element for cisplatin-modified DNA. SSRP1 is also a component of a CK2-SPT16-SSRP1 complex which forms following UV irradiation, comprising SSRP1, SUPT16H, CSNK2A1, CSNK2A2 and CSNK2B. SSRP1 has been shown to interact with NEK9, a serine/threonine-protein kinase. SSRP1 protein also functions as a co-activator of the transcriptional activator p63 (including, for example, isoform gamma of TP63). SSRP1 enhances the ‘activity of full-length p63, but it has no effect on the N-terminus-deleted b63 (DeltaN-p63) variant. SSRP1 also interacts with FYTTD1/UIF and SRF.

SPI16 (SUPT16H) is a protein that in humans is encoded by the SUPT16H gene (mRNA in humans: NM_07192.3, the sequence is hereby incorporated by reference in its entirety, mRNA in mouse: NM_033618.3, the sequence is hereby incorporated by reference in its entirety). The SPT16 protein (in humans: NP_009123.1, the sequence is hereby incorporated by reference in its entirety, in mouse: NP_291096.2, the sequence is hereby incorporated by reference in its entirety) is the 140 kDa subunit in the FACT complex. SPT16 is also a component of a CK2-SPT16-SSRP1 complex which forms following UV irradiation, comprising SSRP1, SUPT16H, CSNK2A1, CSNK2A2 and CSNK2B. Additionally, SPT16 is a component of the WINAC complex, comprising, at least, SMARCA2, SMARCA4, SMARCB1, SMARCC1, SMARCC2, SMARCD1, SMARCE1, ACTL6A, BAZ1B/WSTF, ARID1A, SUPT16H, CHAF1A and TOP2B. SPT16 has been shown to interact with BAZ1B, a tyrosine-protein kinase. SPT16 also interacts with NEK9, general transcription factor IIE subunit 2 (GTF2E2), and binds to histone H2A-H2B.

FACT has been linked to aggressive cancers of many varieties. See U.S. Provisional Application No. 61/763266, filed Feb. 11, 2013, the contents of which are hereby incorporated by reference herein in their entirety.

In some embodiments, FACT levels or activity, more specifically, in some embodiments, the ratio of chromatin-bound FACT to soluble FACT is useful as a companion diagnostic assay. In some embodiments, the ratio of chromatin-bound FACT to soluble FACT is useful to predict a subject's response to a curaxin, including Curaxin 137. In some embodiments, an increased ratio of chromatin-bound FACT to soluble FACT in a subject's sample upon curaxin treatment directs the administering of a curaxin, including Curaxin 137 to the subject. Conversely, in some embodiments, a reduced or unchanged ratio of chromatin-bound FACT to soluble FACT in a subject's sample upon curaxin treatment directs away from the administering of a curaxin, including Curaxin 137 to the subject. In some embodiments, the ratio of chromatin-bound FACT to soluble FACT finds use as a pharmacodynamic assay for curaxin, including Curaxin 137 response.

In some embodiments, the present invention provides for a diagnostic for curaxin response in cancers comprising measuring the ratio of chromatin-bound FACT to soluble FACT in blood. In some embodiments, the present invention provides for a diagnostic for curaxin response in solid tumors comprising measuring the ratio of chromatin-bound FACT to soluble FACT in blood. In some embodiments, the measurement comprises an ELISA.

In various embodiments, the diagnostic techniques described herein comprise a measurement of a ratio of chromatin-bound FACT to soluble FACT in a sample (e.g. blood). In some embodiments, the diagnostic techniques described herein comprise a measurement of baseline or normal levels of the ratio of chromatin-bound FACT to soluble FACT. In some embodiments, this measurement is before or at the administering a curaxin to a subject. In some embodiments, further measurements occur at about 0.1, or about 0.3, or about 0.5, or about 1, or about 1.5, or about 2.0, or about 2.5, or about 3.0, or about 3.5, or about 4, or about 5, or about 6, or about 7, or about 8, or about 9, or about 10 hours after administering a curaxin to a subject. In some embodiments, further measurements occur directly following administering a curaxin to a subject.

In some embodiments, a high ratio of chromatin-bound FACT to soluble FACT directs treatment with curaxins. In some embodiments, a low ratio of chromatin-bound FACT to soluble FACT directs away from treatment with curaxins. In some embodiments, a high ratio of chromatin-bound FACT to soluble FACT is about 3.0, or about 2.75, or about 2.5, or about 2.25, or about 2.0, or about 1.75. In some embodiments, a low ratio of chromatin-bound FACT to soluble FACT is about 0, or about 0.25, or about 0.5.

In various embodiments, the present methods allow for dose determination and regulation via measurement of a ratio of chromatin-bound FACT to soluble FACT in a sample (e.g. blood). For example, a medical practitioner can optimize dose by measuring the levels or activity in a sample (e.g. blood) and altering dose to correlate with a ratio of chromatin-bound FACT to soluble FACT that is reflective of curaxin activity.

In one aspect, the present invention provides a method for treating cancer, comprising administering an effective amount of a curaxin compound including, but not limited to a compound of Formula I to a subject in need thereof, wherein the subject's ratio of chromatin-bound FACT to soluble FACT is elevated to greater than 1.75 after treatment. In some embodiments, the method further comprises measuring the subject's ratio of chromatin-bound FACT to soluble FACT. In some embodiments, the measurement occurs at about 0.1, or about 0.3, or about 0.5, or about 1, or about 1.5, or about 2.0, or about 2.5, or about 3.0, or about 3.5, or about 4, or about 5, or about 6, or about 7, or about 8, or about 9, or about 10 hours after administering a curaxin to a subject. In some embodiments, further measurements occur at about a day, or two days after administering a curaxin to a subject. In some embodiments, the subject's ratio of chromatin-bound FACT to soluble FACT is about 3.0, or about 2.75, or about 2.5, or about 2.25, or about 2.0, or about 1.75., in various embodiments, the measurement is made with blood samples.

In various embodiments, the present invention pertains to a NF-kB-responsive marker (e.g. CXCL10) and/or NF-kB. C-X-C motif chemokine 10 (CXCL10) also known as Interferon gamma-induced protein 10 (IP-10) or small-inducible cytokine B10 is an 8.7 kDa protein that in humans is encoded by the CXCL10 gene. CXCL10 is secreted by several cell types in response to IFN-γ. These cell types include monocytes, endothelial cells and fibroblasts. CXCL10has been attributed to several roles, such as, without wishing to be bound by theory, chemoattraction for monocytes/macrophages, T cells, NK cells, and dendritic cells, promotion of T cell adhesion to endothelial cells, antitumor activity, and inhibition of bone marrow colony formation and angiogenesis. This chernokine elicits its effects by binding to the cell surface chernokine receptor CXCR3. CXCL10 is encoded by NF-kB-regulated gene.

NF-kappaB (NF-kB) proteins comprise a family of structurally-related eukaryotic transcription factors that are involved in the control of a large number of normal cellular and organismal processes, such as immune and inflammatory responses, developmental processes, cellular growth, and apoptosis. In addition, these transcription factors are persistently active in a number of disease states, including cancer, arthritis, chronic inflammation, asthma, neurodegenerative diseases, and heart disease

In various embodiments, the amount of a NF-kB-responsive marker (e.g. CXCL10) and/or NF-kB is useful for directing patient treatment with a curaxin, including Curaxin 137. For example, in some embodiments, the decreased levels or activity of a NF-kB-responsive marker (e.g. CXCL10) and/or NF-kB in a subject's sample upon curaxin treatment indicates bioavailability in a subject and directs the administering of curaxin, including Curaxin 137. Conversely, in some embodiments, an increased or unchanged amount of a NF-kB-responsive marker (e.g. CXCL10) and/or NF-kB in a subject's sample upon curaxin treatment directs away from the administering of a curaxin, including Curaxin 137 to the subject. In some embodiments, the measurement of a NF-kB-responsive marker (e.g. CXCL10) and/or NF-kB finds use as a pharmacokinetic assay for curaxin, including Curaxin 137 response.

In some embodiments, the present invention provides for a diagnostic for curaxin response in cancers comprising measuring a NF-kB-responsive marker (e.g. CXCL10) and/or NF-kB in blood. In some embodiments, the present invention provides for a diagnostic for curaxin response in solid tumors comprising measuring a NF-kB-responsive marker (e.g. CXCL10) and/or NF-kB in blood. In some embodiments, the measurement comprises an ELISA.

In some embodiments, measurement of a NF-kB-responsive marker (e.g. CXCL10) and/or NF-kB in a sample (e.g. blood) is before or at the administering a curaxin to a subject. In some embodiments, further measurements occur at about 0.1, or about 0.3, or about 0.5, or about 1, or about 1.5, or about 2.0, or about 2.5, or about 3.0, or about 3.5, or about 4, or about 5, or about 6, or about 7, or about 8, or about 9, or about 10 hours after administering a curaxin to a subject. In some embodiments, further measurements occur directly following administering a curaxin to a subject.

In various embodiments, the present methods allow for dose determination and regulation via measurement of a NF-kB-responsive marker (e.g. CXCL10) and/or NF-kB in a sample (e.g. blood). For example, a medical practitioner can optimize dose by measuring the levels or activity in a sample (e.g. blood) and altering dose to correlate with a level or activity of NF-kB-responsive marker (e.g. CXCL10) and/or NF-kB that is reflective of curaxin activity.

In one aspect, the present invention provides a method for treating cancer, comprising administering an effective amount of a curaxin compound including, but not limited to a compound of Formula I to a subject in need thereof, wherein the subject's level or activity of a NF-kB-responsive marker (e.g. CXCL10) and/or NF-kB is reduced to about 70% of a predose level after treatment. In some embodiments, the method further comprises measuring the subject's level or activity of a NF-kB-responsive marker (e.g. CXCL 10) and/or NF-kB. In some embodiments, the measurement occurs about 0.1, or about 0.3, or about 0.5, or about 1, or about 1.5, or about 2.0, or about 2.5, or about 3.0, or about 3.5, or about 4, or about 5, or about 6, or about 7, or about 8, or about 9, or about 10 hours after administering a curaxin to a subject. In some embodiments, further measurements occur at about a day, or two days after administering a curaxin to a subject. In some embodiments, the subject's level or activity of a NF-kB-responsive marker (e.g. CXCL10) and/or NF-k6 is reduced to about 60%, or about 65%, or 70%, or about 72%, or about 74%, or about 76%, or about 78%, or about 80% of a predose level after treatment. In various embodiments, the measurement is made with blood samples.

In some embodiments, the biomarker, e.g. NF-kB-responsive marker (e.g. CXCL10) and/or NF-kB, may be substituted or supplanted with one or more of interferon-inducible chemokines Interferon-inducible chemokines, CXCL10/IP-10 (interferon gamma inducible Protein 10) and CXCL9MIG (Monokine Induced by Gamma interferon), CCLB/MCP-2 and IFIT3, the human hornolog of mouse interferon-inducible protein ISG49. Accordingly, in some embodiments, the uses of NF-kB-responsive marker (e.g. CXCL10) and/or NF-kB described herein apply equally to CXCL10/IP-10, CXCL9/MIG, CCL8/MCP-2, and IFIT3.

In some embodiments, IFIT3 is used as a biomarker to predict a cancer patient's responsiveness to CBL0137 and optionally direct treatment with CBL0137. In some embodiments, an increase in levels of IFIT3 is indicative of a clinical response to CBL0137 and optionally direct treatment or further treatment with CBL0137. In some embodiments, IFIT3 levels are increased by about 5-fold, or about 7.5-fold, or about 10 fold, or about 12.5 fold, or about 15 fold.

In general, compositions of the invention include a carbazole compound. Suitable carbazole compounds and methods of making them are described in PCT/US2009/059558 (international Patent Publication No. WO 2010/042445), filed Oct. 5, 2009, the entire disclosure of which is incorporated herein by reference. In certain embodiments, the carbazole is a compound referred to herein as a curaxin.

Curaxins combine two groups of chemical compounds with different structures but with similar molecular mechanism of action on tumor cells. The first group includes derivatives of 9-aminoacridine (including, e.g., the antimalarial drug Acrichine). The second group includes compounds that have a carbazole nucleus. In some embodiments, the carbazole structure can comprise acetyl and/or alkylacyl and/or alkyl ketone substituents. In some embodiments, side chain(s) attached to the carbazole nitrogen atom can comprise a linear or branched alkylamine and may contain secondary and tertiary amino groups.

In various embodiments, the present invention relates to a curaxin of Formula I:

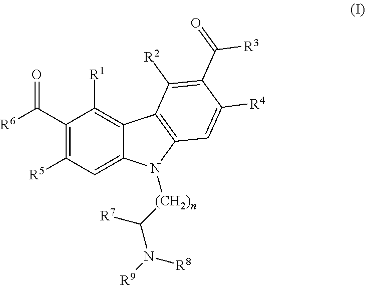

wherein each of R¹-R⁹ are independently H, hydroxyl or alkyl;

n is 0, 1, 2, 3, 4, or 5;

or a pharmaceutically acceptable salt or hydrate thereof.

In one embodiment, the compound of Formula I has the structure:

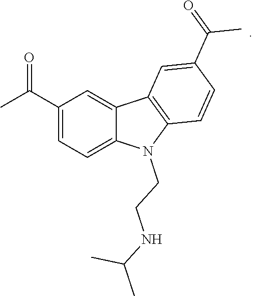 [SMILES: CC(=O)c1ccc2c(c1)c1cc(C(C)=O)ccc1n2CCNC(C)C]

The compound above is referred to generally as a curaxin and specifically referred to herein as curaxin-137 and/or CBL-137 and/or CBLB0137 and/or CBLB137.

In various embodiments, the present invention relates to cancer, including treating or diagnosing it. In some embodiments, the invention pertains to cancer characterized by having a reduced amount of p53 relative to an undiseased state. In some embodiments, the cancer is resistant and/or non-responsive to an agent that targets or effects p53 based on a chemosensitivity test or surrogate biomarker.

A cancer or tumor refers to an uncontrolled growth of cells and/or abnormal increased cell survival and/or inhibition of apoptosis which interferes with the normal functioning of the bodily organs and systems. A subject that has a cancer or a tumor is a subject having objectively measurable cancer cells present in the subject's body. Included in this invention are benign and malignant cancers, as well as dormant tumors or micrometastatses. Cancers which migrate from their original location and seed vital organs can eventually lead to the death of the subject through the functional deterioration of the affected organs.

In various embodiments, the invention is applicable to pre-metastatic cancer, or metastatic cancer. Metastasis refers to the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

Metastases are often detected through the sole or combined use of magnetic resonance imaging (MRI) scans, computed tomography (CT) scans, blood and platelet counts, liver function studies, chest X-rays and bone scans in addition to the monitoring of specific symptoms.

The methods and compositions described herein are directed toward the treatment of cancer, and/or the treatment, prevention or amelioration of growth, progression, and/or metastases of malignancies and proliferative disorders associated with increased cell survival, or the inhibition of apoptosis.

In some embodiments, the cancer is one or more of a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endornetrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer, leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as wet as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome. In some embodiments, the cancer is an advanced solid tumor, optionally originating from lung, kidney, colon as well as head and neck, breast or other organs with at least one measurable lesion.

In some embodiments the curaxin, including but not limited to a compound of Formula I, or a pharmaceutically acceptable salt is administered in combination with an additional cancer therapy, such as, for example, an agent that targets or effects p53. In some embodiments, an agent that targets or effects p53 restores and/or increases wild type activity and/or levels. In some embodiments, an agent that targets or effects p53 restores and/or increases tumor suppression activity, including but not limited to checkpoint inhibition activity. Exemplary agents that targets or effect p53 include, but are not limited to, those that are described in the following, the contents of which are hereby incorporated by reference in their entireties: Lane et al. Cold Spring Harb Perspect Biol 2010; 2:a001222; Wang et al. Transl Oncol. 2010 February; 3(1): 1-12; Chèrie, Expert Opinion on Therapeutic Patents, June 2001, Vol. 11, No. 6, Pages 923-935,

In some embodiments, the cancer is multidrug resistant. For example, the patient may have undergone one or more cycles of chemotherapy, without substantial response. Alternatively or in addition, the tumor has one or more markers of multidrug resistance. Such markers can include chemoresponse assays or molecular assays. Thus the term multidrug resistant includes a cancer that has exhibited non-responsiveness to at least one cycle of combination chemotherapy, or alternatively, has scored (diagnostically) as resistant to at least two of (including comparable agent to) docetaxel, paclitaxel, doxorubicin, epirubicin, carboplatin, cisplatin, vinblastine, vincristine, oxaliplatin, carmustine, fluorouracil, gemcitabine, cyclophosphamide, ifosfamide, topotecan, erlotinib, etoposide, and mitomycin.

In various embodiments, the cancer is resistant to platinum drugs and/or taxanes, or is determined to be resistant to platinum drugs or taxanes based on a chemosensitivity test or surrogate biomarker.

As is known in the art, taxanes are a group of drugs that includes, but is not limited to, paclitaxel (TAXOL) and docetaxel (TAXOTERE). Taxanes, without wishing to be bound by theory, prevent the growth of cancer cells by affecting microtubules, which play an important role in cell function. Platinum (Pt) is a naturally occurring element. Platinum drugs, without wishing to be bound by theory, cause cell death by the formation of chemical cross-links in DNA that interfere with DNA replication and transcription. Platinum drugs are a group of drugs that includes, but is not limited to, cisplatin (PLATINOL), carboplatin (PARAPLATIN), and oxaliplatin (ELOXATIN).

In various embodiments, the curaxin, including but not limited to a compound of Formula I, or a pharmaceutically acceptable salt is administered as an adjuvant therapy after resection, including, without limitation, as the sole adjuvant therapy.

Adjuvant therapy, also called adjuvant care, is treatment that is given in addition to the primary, main or initial treatment. By way of non-limiting example, adjuvant therapy may be an additional treatment usually given after surgery where all detectable disease has been removed, but where there remains a statistical risk of relapse due to occult disease. In some embodiments, a curaxin, including but not limited to a compound of Formula I, or a pharmaceutically acceptable salt thereof is used as an adjuvant therapy in the treatment of cancer, including cancers that are characterized by having reduced p53 relative to a non-cancerous state. In some embodiments, a curaxin, including but not limited to a compound of Formula I, or a pharmaceutically acceptable salt thereof is used as the sole adjuvant therapy in the treatment of cancer, including cancers that are characterized by having reduced p53 relative to a non-cancerous state.

In some embodiments the curaxin, including but not limited to a compound of Formula I, or a pharmaceutically acceptable salt is administered as a neoadjuvant therapy prior to resection.

In certain embodiments, neoadjuvant therapy refers to therapy to shrink and/or downgrade a tumor prior to any surgery. In some embodiments, neoadjuvant therapy means chemotherapy administered to cancer patients prior to surgery. In some embodiments, neoadjuvant therapy means an agent described herein administered to cancer patients prior to surgery. Types of cancers for which neoadjuvant chemotherapy is commonly considered include, for example, breast, colorectal, ovarian, cervical, bladder, and lung. In some embodiments, a curaxin, including but not limited to a compound of Formula I, or a pharmaceutically acceptable salt thereof is used as a neoadjuvant therapy in the treatment of cancer, including the subtypes described herein, prior to resection.

In another aspect, the present invention provides a method for identifying a subject with a tumor that is likely to respond to treatment with a curaxin, including but not limited to a compound of Formula I, comprising evaluating the tumor and/or a blood sample, comprising measuring a presence, absence, level, or activity of p53 and/or a NF-kB-responsive marker (e.g. CXCL10) and/or NF-kB, wherein a reduced level or activity of p53 and/or a NF-kB-responsive marker (e.g. CXCL10) and/or NF-kB relative to a non-tumor state indicates that the subject is likely respond to treatment with a curaxin, including but not limited to a compound of Formula I, and optionally directs administering an effective amount of the curaxin, including but not limited to a compound of Formula I, to a subject that is likely to respond to the curaxin, including but not limited to a compound of Formula I.

In another aspect, the present invention provides a method for identifying a subject with a tumor that is likely to respond to treatment with a curaxin, including but not limited to a compound of Formula I, comprising evaluating the tumor and/or a blood sample, comprising measuring a ratio of chromatin-bound FACT to soluble FACT, wherein an increased ratio of chromatin-bound FACT to soluble FACT relative to a non-tumor state indicates that the subject is likely respond to treatment with a curaxin, including but not limited to a compound of Formula I, and optionally directs administering an effective amount of the curaxin, including but not limited to a compound of Formula I, to a subject that is likely to respond to the curaxin, including but not limited to a compound of Formula I.

In another aspect, the present invention provides a method for a method for identifying a subject with a tumor that is likely to respond to treatment with an agent that targets or effects p53, comprising administering an effective amount of a curaxin, including but not limited to a compound of Formula I, and evaluating the tumor and/or a blood sample, comprising measuring a presence, absence, level, or activity of p53, wherein an increased level or activity of p53 relative to an untreated state indicates that the subject is likely respond to an agent that targets or effects p53.

In some embodiments, the methods useful for evaluating diagnosis, prognosis, and response to treatment are related to those U.S. Provisional Application No. 61/763,266, filed Feb. 11, 2013, the contents of which are hereby incorporated by reference herein in their entirety

Diagnosis refers to the process of attempting to determine or identify a possible disease or disorder, such as, for example, cancer. Prognosis refers to the predicting of a likely outcome of a disease or disorder, such as, for example, cancer. A complete prognosis often includes the expected duration, the function, and a description of the course of the disease, such as progressive decline, intermittent crisis, or sudden, unpredictable crisis. Response to treatment is a prediction of a patient's medical outcome when receiving a treatment. Responses to treatment can be, by way of non-limiting example, pathological complete response, survival, and probability of recurrence.

In various aspects, the present invention comprises evaluating a tumor and/or blood sample. In various embodiments, the evaluation may be selected from diagnosis, prognosis, and response to treatment. In various embodiments, the present methods allow for dose determination and regulation.

In various embodiments, the measurement comprises evaluating a presence, absence, or level of a protein. In another embodiment, the measurement comprises evaluating a presence, absence, or level of expression of a nucleic acid.

In still other embodiments, the measurement comprises contacting a specimen of the tumor or cells cultured from the tumor with an agent that specifically binds p53 and/or a NF-kB-responsive marker (e.g. CXCL10) and/or NF-kB and/or FACT, including, but not limited to, an antibody.

In still other embodiments, the measurement of p53 and/or a NF-kB-responsive marker (e.g. CXCL10) and/or NF-kB and/or FACT comprises one or more of immunohistochemical staining, western blotting, in cell western, immunofluorescent staining, ELISA, and fluorescent activating cell sorting (FACS), or any other method described herein or known in the art.

Methods of an the invention may involve contacting an antibody (e.g. against p53 and/or a NF-kB-responsive marker (e.g. CXCL10) and/or NF-kB and/or FACT) with a tumor specimen (e.g. biopsy or tissue or body fluid) in order to identify an epitope that is specific to the tissue or body fluid and that is indicative of a state of a cancer (e.g. against p53 and/or a NF-RB-responsive marker (e.g. CXCL10) and/or NF-kB and/or FACT).

There are generally two strategies used for detection of epitopes on antigens in body fluids or tissues, direct methods and indirect methods. The direct method comprises a one-step staining, and may involve a labeled antibody (e.g. FITC conjugated antiserum) reacting directly with the antigen in a body fluid or tissue sample. The indirect method comprises an unlabeled primary antibody that reacts with the body fluid or tissue antigen, and a labeled secondary antibody that reacts with the primary antibody. Labels can include radioactive labels, fluorescent labels, hapten labels such as, biotin, or an enzyme such as horse radish peroxidase or alkaline phosphatase. Methods of conducting these assays are well known in the art. See. e.g., Harlow et al. (Antibodies, Cold Spring Harbor Laboratory, NY, 1988), Harlow et al. (Using Antibodies, A Laboratory Manual, Cold Spring Harbor Laboratory, NY, 1999), Virella (Medical Immunology, 6th edition, Informa HealthCare, New York, 2007), and Diamandis at al. (Immunoassays, Academic Press, Inc., New York, 1996). Kits for conducting these assays are commercially available from, for example, Clontech Laboratories, LLC. (Mountain View, Calif.).

In various embodiments, antibodies include whole antibodies and/or any antigen binding fragment (e.g., an antigen-binding portion) and/or single chains of these (e.g. an antibody comprising at least two heavy (H) chains and two light (L) chains inter-connected by disulfide bonds, an Fab fragment, a monovalent fragment consisting of the V_{L}, V_{H}, C_{L} and CH1 domains; a F(ab)₂ fragment, a bivalent fragment including two Fab fragments linked by a disulfide bridge at the hinge region; a Fd fragment consisting of the V_{H} and CH1 domains; a Fv fragment consisting of the V_{L} and V_{H} domains of a single arm of an antibody; and the like). In various embodiments, polyclonal and monoclonal antibodies are useful, as are isolated human or humanized antibodies, or functional fragments thereof.

Standard assays to evaluate the binding ability of the antibodies toward the target of various species are known in the art, including for example, ELISAs, western blots and RIAs. The binding kinetics (e.g., binding affinity) of antibodies also can be assessed by standard assays known in the art, such as by Biacore analysis.

In another embodiment, the measurement comprises evaluating a presence, absence, or level of a nucleic acid.

A person skilled in the art will appreciate that a number of methods can be used to detect or quantify the DNA/RNA levels or activity of p53 and/or a NF-kB-responsive marker (e.g. CXCL10) and/or NF-kB and/or FACT.

Gene expression can be measured using, for example, low-to-mid-plex techniques, including but not limited to reporter gene assays, Northern blot, fluorescent in situ hybridization (FISH), and reverse transcription PCR (RT-PCR). Gene expression can also be measured using, for example, higher-plex techniques, including but not limited, serial analysis of gene expression (SAGE), DNA microarrays. Tiling array, RNA-Seq/whole transcriptome shotgun sequencing (WTSS), high-throughput sequencing, multiplex PCR, multiplex ligation-dependent probe amplification (MLPA), DNA sequencing by ligation, and Luminex/XMAP. In one embodiment, the levels or activity of the biomarkers disclosed herein are measured using sequencing, including, for example determining amounts of wild type and mutant biomarkers.

A person skilled in the art will appreciate that a number of methods can be used to detect or quantify the level of RNA products of the biomarkers within a sample, including arrays, such as microarrays, RT-PCR (including quantitative PCR), nuclease protection assays and Northern blot analyses.

In some embodiments, the present invention includes the measurement of a tumor specimen, including biopsy or surgical specimen samples. In some embodiments, the biopsy is a human biopsy. In various embodiments, the biopsy is any one of a frozen tumor tissue specimen, cultured cells, circulating tumor cells, and a formalin-fixed paraffin-embedded tumor tissue specimen.

In some embodiments, the tumor specimen may be a biopsy sample, such as a frozen tumor tissue (cryosection) specimen. As is known in the art, a cryosection may employ a cryostat, which comprises a microtome inside a freezer. The surgical specimen is placed on a metal tissue disc which is then secured in a chuck and frozen rapidly to about −20° C. to about −30° C. The specimen is embedded in a gel like medium consisting of, for example, poly ethylene glycol and polyvinyl alcohol. The frozen tissue is cut frozen with the microtome portion of the cryostat, and the section is optionally picked up on a glass slide and stained.

In some embodiments, the tumor specimen may be a biopsy sample, such as cultured cells. These cells may be processed using the usual cell culture techniques that are known in the art. These cells may be circulating tumor cells.

In some embodiments, the tumor specimen may be a biopsy sample, such as a formalin-fixed paraffin-embedded (FFPE) tumor tissue specimen. As is known in the art, a biopsy specimen may be placed in a container with formalin (a mixture of water and formaldehyde) or some other fluid to preserve it. The tissue sample may be placed into a mold with hot paraffin wax. The wax cools to form a solid block that protects the tissue. This paraffin wax block with the embedded tissue is placed on a microtome, which cuts very thin slices of the tissue.

In certain embodiments, the tumor specimen contains less than 100 mg of tissue, or in certain embodiments, contains about 50 mg of tissue or less. The tumor specimen (or biopsy) may contain from about 20 mg to about 50 mgs of tissue, such as about 35 mg of tissue.

The tissue may be obtained, for example, as one or more (e.g., 1, 2, 3, 4, or 5) needle biopsies (e.g., using a 14-gauge needle or other suitable size). In some embodiments, the biopsy is a fine-needle aspiration in which a long, thin needle is inserted into a suspicious area and a syringe is used to draw out fluid and cells for analysis. In some embodiments, the biopsy is a core needle biopsy in which a large needle with a cutting tip is used during core needle biopsy to draw a column of tissue out of a suspicious area. In some embodiments, the biopsy is a vacuum-assisted biopsy in which a suction device increases the amount of fluid and cells that is extracted through the needle. In some embodiments, the biopsy is an image-guided biopsy in which a needle biopsy is combined with an imaging procedure, such as, for example, X ray, computerized tomography (CT), magnetic resonance imaging (MRI) or ultrasound. In other embodiments, the sample may be obtained via a device such as the MAMMOTOME® biopsy system, which is a laser guided, vacuum-assisted biopsy system for breast biopsy.

In some embodiments, the present invention provides for the methods for treatment as described herein in which a curaxin, including but not limited to a compound of Formula I, or its pharmaceutically acceptable salt may be combined with other anti-cancer treatment modalities. These modalities may include, for example, surgical resection, radiation therapy (including the use of the compounds as described herein as, or in combination with, radiosensitizing agents), chemotherapy, pharmacodynamic therapy, targeted therapy, immunotherapy, and supportive therapy (e.g., painkillers, diuretics, antidiuretics, antivirals, antibiotics, nutritional supplements, anemia therapeutics, blood clotting therapeutics, bone therapeutics, and psychiatric and psychological therapeutics). Such other anti-cancer treatments may be provided sequentially (e.g., before or after) or simultaneously with the administration of a curaxin, including but not limited to a compound of Formula I, or its pharmaceutically acceptable salt.

In some embodiments, the present invention provides for the methods for treatment as described herein with a curaxin, including but not limited to a compound of Formula I, or its pharmaceutically acceptable salt, and an additional therapeutic, or its pharmaceutically acceptable salt. In some embodiments, the present invention provides for compositions comprising a curaxin, including but not limited to a compound of Formula I, or its pharmaceutically acceptable salt, and an additional therapeutic, or its pharmaceutically acceptable salt. In other embodiments, the invention provides for the use of curaxins, including but not limited to compounds of Formula I, or their pharmaceutically acceptable salts, alone or in combination with an additional therapeutic or their pharmaceutically acceptable salts, in the manufacture of a medicament useful for the treatment or prevention of one or more cancers.

Such additional therapeutic may be provided sequentially (e.g., before or after) or simultaneously with the administration of a curaxin, including but not limited to a compound of Formula I, or its pharmaceutically acceptable salt. In some embodiments, a curaxin, including but not limited to a compound of Formula I, or its pharmaceutically acceptable salt may be conjugated to an additional therapeutic.

In embodiments involving conjugation, such conjugates can enhance delivery of the compounds to a particular anatomical site or region of interest (e.g., a tumor), enable sustained therapeutic concentrations of the compounds in target cells, alter pharmacokinetic and pharmacodynamic properties of the compounds, and/or improve the therapeutic index or safety profile of the compounds. Suitable auxiliary moieties include, for example, amino acids, oligopeptides, or polypeptides, e.g., antibodies such as monoclonal antibodies and other engineered antibodies; and natural or synthetic ligands to receptors in target cells or tissues. Other suitable auxiliaries include fatty acid or lipid moieties that promote biodistribution and/or uptake of the compound by target cells (see, e.g., Bradley et al., Clin. Cancer Res. (2001) 7:3229).

In exemplary embodiments, the invention provides various additional therapeutics. Examples of additional therapeutics include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone), a camptothecin (including the synthetic analogue topotecan); bryostatin; cally statin; CC-1065 (including its adozelesin, carzelesin and bizelesin synthetic analogues); cryptophycins cryptophycin 1 and cryptophycin 8); dolastatin; duocarmycin (including the synthetic analogues, KW-2189 and CB 1-TM1); eleutherobin, pancratistatin; a sarcodictyin; spongistatin; nitrogen mustards such as chlorambucil, chlomaphazine, cholophosphamide, estramustine, Ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnusfine; antibiotics such as the enediyne antibiotics (e.g., calicheamicin, especially calicheamicin gammall and calicheamicin ornegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinornysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonignin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolidc acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above.

Many cancer treatment protocols currently employ radiosensitizers activated by electromagnetic radiation, e.g., X-rays. The present invention also provides for the use of such compounds as additional therapeutics. Examples of X-ray-activated radiosensitizers include, but are not limited to, metronidazole, misonidazole, desmethylmisonidazole, pimonidazole, etanidazole, nimorazole, mitomycin C, RSU 1069, SR 4233, EO9, RB 6145, nicotinamide, 5-bromodeoxyuridine (BUdR), 5-iododeoxyuridine (lUdR), bromodeoxycytidine, fluorodeoxyuridine (FUdR), hydroxyurea, cis-platin, and therapeutically effective analogs and derivatives of the same.

Photodynamic therapy (PDT) employs visible light as the radiation activator of the sensitizing agent. The present invention also provides for the use of such compounds as additional therapeutics. Examples of photodynamic radiosensitizers include, but are not limited to hematoporphyrin derivatives, PHOTOFRIN®, benzoporphyrin derivatives, NPe6, tin etioporphyrin (SnET2), pheoborbide-a, bacteriochlorophyll-a, naphthalocyanines, phthalocyanines, zinc phthalocyanine, and therapeutically effective analogs and derivatives of the same.

Any agent described herein can possess a sufficiently basic functional group, which can react with an inorganic or organic add, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in Journal of Pharmaceutical Science, 66, 2-19 (1977) and The Handbook of Pharmaceutical Salts; Properties, Selection, and Use. P. H. Stahl and C. G. k, Aiermuth (ads.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chiorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term phamiaceutically acceptable salt also refers to a salt of the compounds of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkyiamines, dicyclohexylamine; tributyl amine; pyridine; N-methyi, N-ethyiamine; diethylamine; triethylarnine; mono-, bis-, tris-(2-OH-lower alkylarnines), such as mono-; bis-, or tris-(2-hydroxyethyl)arnine. 2-hydroxy-tert-butylamine, or tris-(hydroxymethyl)methylamine, N,N-di-lower (hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In some embodiments, a representative pharmaceutically acceptable salt includes, e.g., water-soluble and water-insoluble salts, such as the acetate, amsonate (4,4-diaminostilbene-2,2-disulfonate), benzenesulfonate, benzonate, bicarbonate, bisulfate, bitartrate., borate, bromide, butyrate, calcium, calcium edetate, camsyiate, carbonate, chloride, citrate, clavulariate, dihydrochloride, edetate, edisyiate, estoiate, esyiate, fiunarate, gluceptate, gluconate, glutamate, glycollylarsanilate, hexafluorophosphate, hexyiresorcinate, hydrabamine, hydrobromide, hydrochloride, hydroxynaphthoate, iodide, isothionate, lactate, lactobionate., laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphateldiphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide and valerate salts.

Further, any agent described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

Pharmaceutical excipients can be liquids, such as water and oils, including those of petroleum, animal, vegetable, or synthetic origin, such as peanut of, soybean oil, mineral oil, sesame oil and the like. The pharmaceutical excipients can be saline, gum acacia, gelatin, starch paste, talc, keratin, colloidal silica, urea and the like. In addition, auxliary, stablizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

Any agent described herein can take the form of solutions, suspensions, emulsion, tablets, pills, pellets, capsules, capsules containing liquids, powders, sustained-release formulations, suppositories, emulsions, aerosols, sprays, suspensions, or any other form suitable for use. Other examples of suitable pharmaceutical excipients are described in Remington's Pharmaceutical Sciences 1447-1676 (Alfonso R. Gerinaro eds., 19th ed. 1995), incorporated herein by reference.

In one embodiment, any agent described herein is formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any agent described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade.

The ingredients may be supplied either separately or mixed together in unit dosage form, for example, as a pre-mixed solution, dry lyophilized-powder, or water-free concentrate in a hermetically sealed container such as an ampule, pre-filled syringe, or sachette indicating the quantity of active agent. Where any agent described herein is to be administered by infusion, it can be dispensed, for example, with an infusion bottle containing sterile pharmaceutical grade water or saline. Where any agent described herein is to be administered by injection, an ampule of sterile water for injection or saline can be provided so that the ingredients can be mixed prior to administration.

Any agent described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or ether physiological conditions or compounds.

Compositions can be prepared according to conventional mixing, granulating, coating or polymerization methods, respectively, and the present compositions can comprise, in one embodiment, from about 0.1% to about 99%; and in another embodiment from about 1% to about 70% of any agent described herein by weight or volume.

In another embodiment, any agent described herein acts synergistically when co-administered with another agent and is administered at doses that are lower than the doses commonly employed when such agents are used as monotherapy. For example, in some embodiments, a curaxin, including but not limited to a compound of Formula I, or its pharmaceutically acceptable salt and an additional therapeutic, or its pharmaceutically acceptable salt can have greater than additive effects when administered in combination or conjugation. In some embodiments, a curaxin, including but not limited to a compound of Formula I or its pharmaceutically acceptable salt and an additional therapeutic, or its pharmaceutically acceptable salt can have synergistic effects when administered in combination or conjugation.

For example, the dosage any agent described herein as wet as the dosing schedule can depend on various parameters, including, but not limited to, the cancer being treated, the subject's general health, and the administering physician's discretion. Any agent described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional therapeutic agent, to a subject in need thereof. In various embodiments any agent described herein is administered 1 minute apart, 10 minutes apart, 30 minutes apart, less than 1 hour apart, 1 hour apart, 1 hour to 2 hours apart, 2 hours to 3 hours apart_{;} 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart.

The amount of any agent described herein that is admixed with the carrier materials to produce a single dosage can vary depending upon the subject being treated and the particular mode of administration. In vitro or in vivo assays can be employed to help identify optimal dosage ranges.

In general, the doses that are useful are known to those in the art. For example, doses may be determined with reference Physicians' Desk Reference, 66th Edition, PDR Network; 2012 Edition) (Dec. 27, 2011), the contents of which are incorporated by reference in its entirety.

The dosage of any agent described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administrat on, the nature of the formulation, the rate of excretion, the particular cancer being treated, the severity of the disorder, and the anatomical location of the disorder. Some variations in the dosage can be expected.

Generally, when orally administered to a mammal, the dosage of any agent described herein may be 0.001 mg/kg/day to 100 mg/kg/day, 0.01 mg/kg/day to 50 mg/kg/day, or 0.1 mg/kg/day to 10 mg/kg/day. When orally administered to a human, the dosage of any agent described herein is normally 0.001 mg to 1000 mg per day, 1 mg to 600 mg per day, or 5 rug to 30 mg per day. In one embodiment, oral dosage is 600 mg per day. In one embodiment, the oral dosage is two 300 mg doses per day. In another embodiment, oral dosage is 7.5 mg per week to 15 mg per week.

For administration of any agent described herein by parenteral injection, the dosage is normally 0.1 mg to 250 rug per day, 1 rug to 20 mg per day, or 3 mg to 5 mg per day. Injections may be given up to four times daily. Generally, when orally or parenterally administered, the dosage of any agent described herein is normally 0.1 mg to 1500 mg per day, or 0.5 rug to 10 mg per day, or 0.5 mg to 5 mg per day. A dosage of up to 3000 mg per day can be administered.

In some embodiments, non-limiting examples of the dose of a curaxin, including but not limited to a compound of Formula I, or its pharmaceutically acceptable salt and an additional therapeutic or its pharmaceutically acceptable salt (in combination or administered individually) may be in a range of about 0.1 mg/kg to about 100 mg/kg of body weight of the subject, for example, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 20 mg/ke_{d}, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg body weight, inclusive of all values and ranges therebetween.

Routes of administration may include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin.

The mode of administration can be left to the discretion of the practitioner, and depends in-part upon the site of the medical condition. In most instances, administration results in the release of any agent described herein into the bloodstream.

Any agent described herein can be administered orally. Such agents can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposornes, microparticles, microcaosules, capsules, etc., and can be used to administer.

In specific embodiments, it may be desirable to administer locally to the area in need of treatment.

In another embodiment, delivery can be in a vesicle, in particular a liposome (see Langer, 1990, Science 249:1527-1533: Treat at al., in Liposomes in the Therapy of Infectious Disease and Cancer, Lopez-Berestein and Fidler (eds), Liss, New York, pp. 353-365 (1989). In yet another embodiment, delivery can be in a controlled release system. In one embodiment, a slow release device may be used. In some embodiments, this device consists of a locally delivered erodible or non-erodable liquid, gel, polymer, etc.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, N.Y. (1984); Ranger and Peppas, 1983, J. Macromoi. Sci, Rev. Macromoi. Chem. 23:61; see also Levy et al., 1985. Science 228;190; During et al., 1989, Ann. Neuroi, 25:351; Howard etal. 1989. J. Neurosurg, 71:105). In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of any agent described herein can, independently, be one to four times daily or one to four times per month or one to six times per year or once every two, three, four or five years.

Administration can be for the duration of one day or one month, two months, three months, six months, one year, two years, three years, and may even be for the life of the subject. Chronic, long-term administration may be indicated in some cases. The dosage may be administered as a single dose or divided into multiple doses. In general, the desired dosage should be administered at set intervals for a prolonged period, usually at least over several weeks or months, although longer periods of administration of several months or years or more may be needed.

The dosage regimen utilizing any agent described herein can be selected in accordance with a variety of factors including type, species, age, weight, sex and medical condition of the subject; the severity of the condition to be treated; the route of administration; the renal or hepatic function of the subject; the pharmacogenomic makeup of the individual: and the specific compound of the invention employed. Any agent described herein can be administered in a single daily dose, or the total daily dosage can be administered in divided doses of two, three or four times daily. Furthermore, any agent described herein can be administered continuously rather than intermittently throughout the dosage regimen.

The dosage administered is an effective amount of the agent. Effective amounts, toxicity, and therapeutic efficacy can be determined by standard pharmaceutical procedures in cell cultures or experimental animals, including but not limited to, determining the LD₅₀ (the dose lethal to about 50% of the population) and/or the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD_{50/}ED₅₀. Compositions and methods that exhibit large therapeutic indices may be selected for use, in some embodiments.

A therapeutically effective dose can be estimated initially from cell culture assays. Also, a dose can be formulated in animal models to achieve a circulating plasma concentration range that includes the IC₅₀ (e,g., the concentration of a curaxin, including but not limited to a curaxin, including but not limited to a compound of Formula I. and/or additional therapeutic or a pharmaceutically acceptable salt thereof), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

For example, the potency a curaxin, including but not limited to a compound of Formula I or a pharmaceutically acceptable salt thereof may be evaluated by measuring an ability of the compound to inhibit NE-kB activity or to activate p53. Activation of p53 may be measured using a dose-response assay in which a sensitive assay system is contacted with a compound of interest over a range of concentrations, including concentrations at which no or minimal effect is observed, through higher concentrations at which partial effect is observed, to saturating concentrations at which a maximum effect is observed. The curve also theoretically passes through a point at which the concentration is sufficient to increase activity to a level that is 50% that of the difference between a baseline and the maximal activity in the assay—the EC₅₀ value. Determination of an EC₅₀ value is made using conventional biochemical (acellular) assay techniques or cell-based assay techniques.

Comparisons of the efficacy of activators often are provided with reference to comparative EC,₅₀ values, wherein a higher EC₅₀ indicates that the test compound is less potent, and a lower EC₅₀ indicates that the compound is more potent, than a reference compound. Compounds of the present invention exhibit unexpectedly good potency, e.g p53 activation, in a luciferase reporter cell line assay.

In some embodiments, the effect will result in a quantifiable change of at least about 10%, preferably at least about 20%, about 30%, about 50%, about 70%, or about 90% or more. Therapeutic benefit also includes halting or slowing the progression of the underlying disease or disorder, regardless of whether improvement is realized.

In certain embodiments, an effective amount that will treat cancer will modulate the symptoms typically by at least about 10%, at least about 20%, at least about 30%, at least about 40%, or at least about 50%. In exemplary embodiments, such modulations will result in, for example, statistically significant and quantifiable changes in the numbers of cancerous cells. In some embodiments, this may be a decrease in the numbers of micrometastases in distant organs, a decrease in recurrent metastatic disease, etc.

This invention is further illustrated by the following non-limiting examples.

### EXAMPLES

### Example 1

### Curaxins Effect Various Biomarkers in Human Cancer Patients

The methods employed herein are known in the art. Details of some of these methods are provided below.

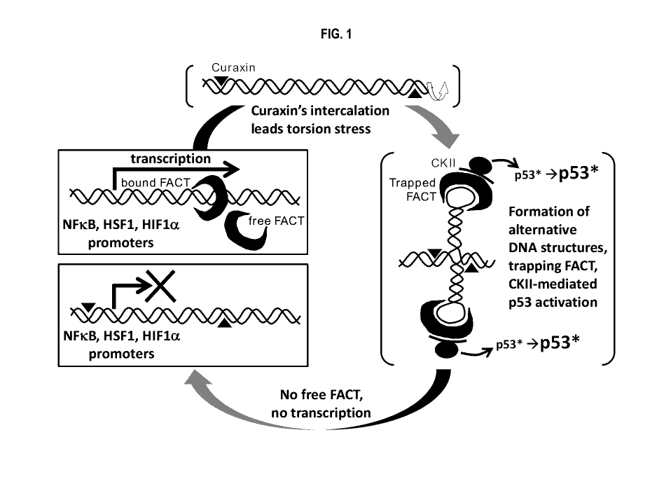

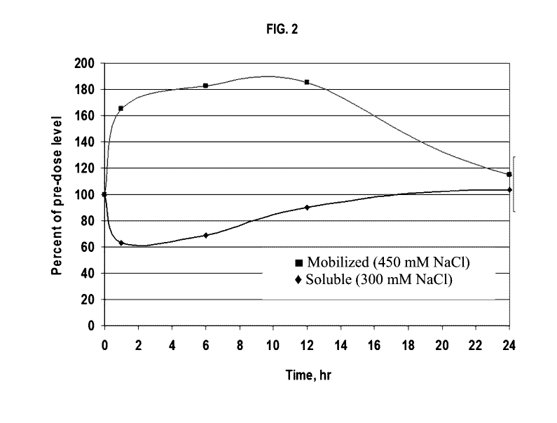

PBMCs of a human patient treated with Curaxin 137 were analyzed by ELISA, FIG. 2 shows the effect of Curaxin 137 on the ratio between bound FACT and free FACT as assayed by ELISA. FACT decreases in soluble fraction and increases in chromatin-mobilize.d (i.e chromatin bound) fraction within first hour after administration of Curaxin 137 in PBMCs of a human patient, Accordingly, the FACT subunit state is indicative of drug activity (FIG. 1).

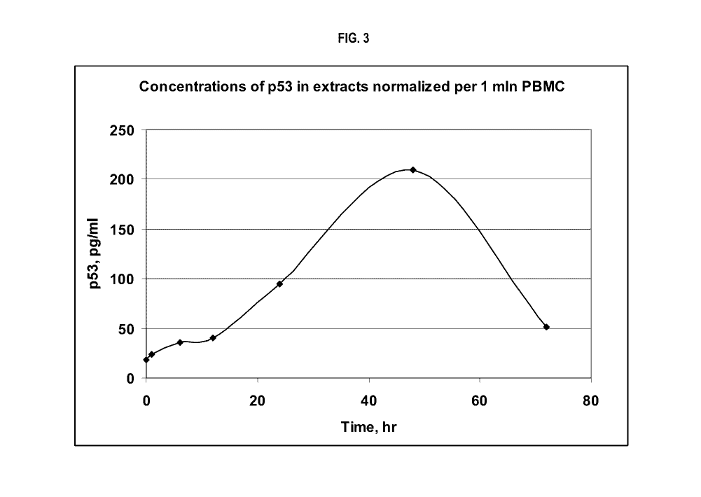

FIG. 3 shows the effect of Curaxin 137 on expression of p53 in peripheral blood mononuclear cells (PBMC) in a human patient. Levels of p53 increase steadily in PBMC during first 48 hours in a human patient.

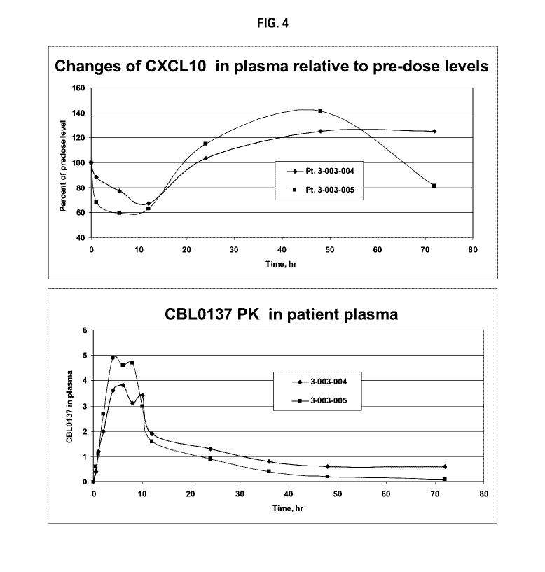

FIG. 4 shows the effect of curaxins NF-kB suppression. CXCL10 is encoded by NF-kB-regulated gene. A sharp drop in CXCL10 blood concentration (ELISA data) was observed after Curaxin 137 administration. This drop in OXCL10 blood concentration correlates with the peak of drug concentration in blood (PK) in two analyzed human patients.

### Example 2

### Biomarker Analysis of Phase 1b Samples

A Phase 1b single agent, dose escalation study of oral administration of CBL0137 in patients with advanced solid tumors that are resistant or refractory to standard of care treatment is ongoing. This is a first-in-human, open-label, non-randomized, multicenter study of CBL0137. Clinical development program starts from Phase 1b in patients with progressive solid tumors as its use. Subjects with any advanced solid tumors preferably originating from lung, kidney, colon as well as head and neck, breast or other organs with at least one measurable lesion of the disease are included in the study.

While the Phase 1b is ongoing, clinical samples from various cohorts were evaluated for various biomarkers.

Sixty three PBMC samples from 6 patients from oral administration CBL0137 trial Cohorts 3 and 4 were analyzed for SSRP1 and p53 using quantitative ELISA. Nuclei, protein and DNA were measured in PBMC in efforts to properly normalize p53 and SSRP1 assay. results for sample variability. Nuclei counts which were considered a good approximation to the original cell numbers, which were underestimated in particularly difficult samples due to poor recovery from pellet clumps. Protein amounts were less variable within sample sets and could be more suitable for data normalization than nuclei counts; however contaminating red blood cells in PBMC contributes to total protein.

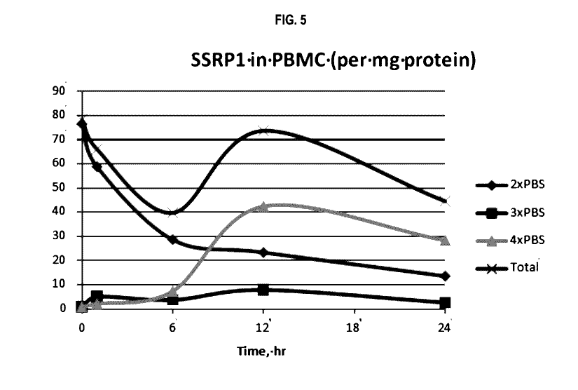

SSRP1 was detected and measured in all test samples and depletion of soluble SSRP1 after administration of CBL0137 was observed, with some variability in the extent and timing of the changes as expected in human data. In two cohort 3 patients, the levels of soluble SSRP1 declined sharply within 6 hours and remained low over extended period of time, In another patient, of cohort 4. SSRP1 also decreased rapidly after administration of CBL0137 and then gradually restored to a predose level over 24 hours. A representative data set is shown in FIG. 5.

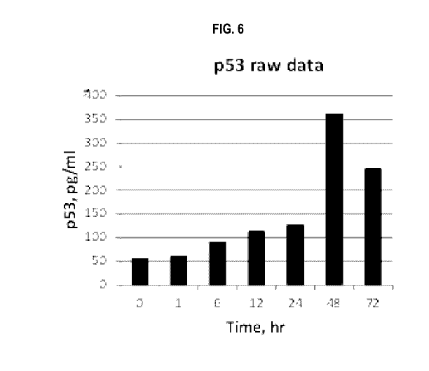

Detectable amounts of p53 were measured in all samples from 3 out of 6 patients; in these PBMC sets, p53 levels increased after administration of CBL0137, reaching a maximum at 24 hours to 48 hours and were about 5 to 10 fold higher than the baseline level. A representative data set is shown in FIG. 6. In one patient, quantifiable levels of p53 were significantly higher at 12 hours and 72 hours.

Levels of SSRP1 and p53 were also measured in 75 PBMC samples from patients in cohorts 5 and 6. The measurements were normalized for estimated cell numbers which were determined by automated nuclei counting and for total protein in the PBMC extracts. To detect and quantitate the distribution of FACT complex between soluble and mobilized state after exposure to CBL0137, SSRP1 was measured in PBMC fractions which were prepared by sequential extraction with a mild detergent and increasing salt concentrations to release nuclear proteins. Depletion of soluble SSRP1 within first hours after administration of CBL0137 was detected in one of the PBMC sets. In efforts to measure the mobilized protein, PBMC fractions which were extracted with a higher salt concentration were analyzed and accumulation of SSRP1 was detected at 6 hours and 12 hours time points in sample sets.

Elevated levels of p53 were detected in four out of six patient PBMC sample sets. The maximum p53 concentrations were 2.6 to 18.5 fold higher than the detection limit and were observed at 12 and 48 hours except for one patient's sample set, demonstrating a maximum at Cycle 2 Day 14 time point.

The assay data for 12 PBMC sets from Cohorts 3 to 6 were analyzed for potential correlation between the individual levels of SSRP1 and p53. A positive correlation was found within this limited number of sample sets between the mean SSRP1 concentrations and Cmax of suggesting that the extent of p53 response to CBL0137 administration could be modulated by the patient's levels of SSRP1.

132 plasma samples from 13 patients participated in CBL0137 oral administration trial (Cohorts 1, 2, 3 and 4) were analyzed for levels of interferon-inducible chemokines interferon-inducible chemokines, CXCL10/IP-10 (interferon gamma inducible Protein 10) and CXCL9/MIG (Monokine Induced by Gamma interferon) and CCL8/MCP-2 to evaluate these proteins as PD biomarkers. The concentrations of chemokines in plasma were measured using improved ELISA methods based on R&D Systems DuoSets to detect CXCL10/IP-10 and CCL8/MCP-2 at 4 pg/mL and CXCL9/MIG at 10 pg/mL.

CXCL10/IP-10 was detected in plasma at variable individual baseline levels with the average values ranging from 8 to 183 pg/mL which were in the range of concentrations found in normal donors (5 to 268 pg/mL, average 107 pg/mL, 40 donors tested); however, CXCL10 in 6 of 13 patients was measured at the levels, corresponding to the low percentile of normal donor distribution (8 to 26 pg/mL). CXCL9/MIG levels correlated with CXCL10 and were found low in the patients with the lower CXCL10 levels and were elevated when CXCL10 was higher, ranging from 13 to 440 pg/mL. The levels of CCL8/MCP-2 in plasma demonstrated a good correlation with both. CXCL10 and CXCL9; however the concentrations were lower and often below the limit of detection. Since concentrations of chemokines varied greatly between patients the relative levels were compared as percentage of the predose concentration.

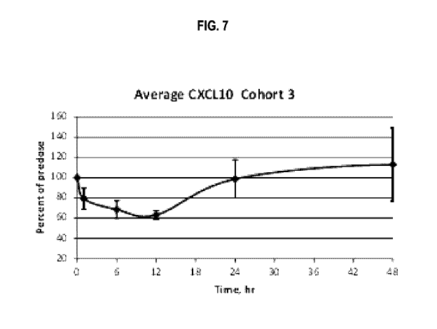

CXCL10/IP-10 was first in various human patient sample sets and it was found that the chemokine levels decreased by 30 to 60% within first hours after CBL0137 administration and then returned to baseline levels, reaching or exceeding predose plasma concentrations at 24 to 48 hours post-exposure. See FIG. 7. Compared with CBL0137 PK profile, such patterns of changes represent an inverse relationship between CBL0137 levels and the chemokine concentrations. Similarly, declines in CXCL10 plasma concentrations were found in other patients.

Plasma levels of IFIT3, the human homolog of mouse interferon-inducible protein ISG49, were evaluated in 75 samples from Cohort 5 and 6 patients using a capture ELISA method which was previously validated using interferon-alpha treated HeLa cell extracts. Due to lack of commercially available IFIT3 protein standard the results were presented as fluorescence measurements (RFU) generated in the same experiment and the relative plasma IFIT3 levels were compared as percentages of the pre-treatment baseline measurements.

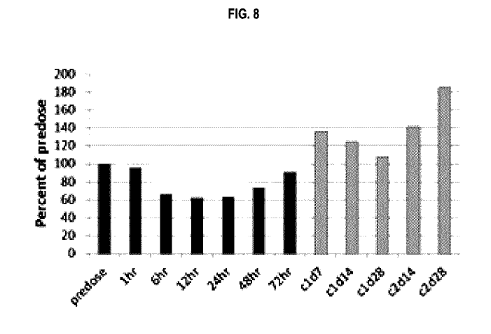

In two patients, IFIT3 varied approximately 3-fold between the minimum and maximum values. The IFIT3 levels in one patient gradually increased, starting with Cycle 1 Day 7 and reaching about 80% higher level at Cycle 2 Day 28 as compared to predose (FIG. 8). In another human patient, about 2-fold IFIT3 increase was observed within a PD analysis time period between 12 hours and 72 hours after CBL0137 administration. In the earner study, rising IFIT3 levels were found in one patient. The data suggest that elevated IFIT3 levels correlate with efficacy of CBL0137 treatment.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A method for treating cancer, comprising administering an effective amount of a curaxin compound or a pharmaceutically acceptable salt or hydrate thereof to a subject in need thereof wherein the cancer is characterized by having reduced levels or activity of p53 relative to a non-cancerous state.

2. The method of claim 1, wherein the curaxin is a compound of Formula I 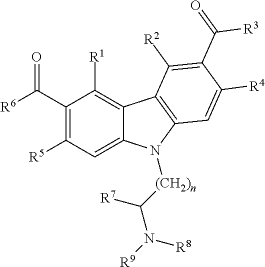
wherein:
each of F¹-R⁹ are independently H, hydroxyl or alkyl;
n is 0, 1, 2, 3, 4, or 5;
or a pharmaceutically acceptable salt or hydrate thereof.

3. The method of claim 1 or 2, wherein the curaxin is administered in combination with an additional therapy.

4. The method of claim 3, wherein the additional therapy is an agent that targets or effects p53.

5. The method of any of the above claims, wherein the curaxin is administered as an adjuvant therapy after resection.

6. The method of any of the above claims, wherein the curaxin is the sole adjuvant therapy.

7. The method of any of the above claims, wherein the curaxin is administered as an adjuvant therapy prior to resection.

8. The method of any of the above claims, wherein the subject is a human.

9. The method of any of the above claims, wherein the cancer is resistant and/or non-responsive to an agent that targets or effects p53 based on a chemosensitivity test or surrogate biomarker.

10. A method for increasing levels or activity of p53, comprising administering an effective amount of a curaxin compound or a pharmaceutically acceptable salt or hydrate thereof to a subject in need thereof;
wherein the level or activity of p53 in blood is increased greater than at least 5-fold relative to an untreated state.

11. The method of claim 10, wherein the curaxin is a compound of Formula I 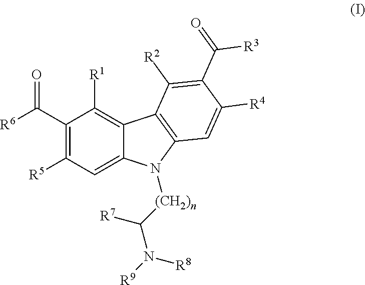
wherein:
each of R¹-R.⁹ are independently H, hydroxyl or alkyl;
n is 0, 1, 2, 3, 4, or 5;
or a pharmaceutically acceptable salt or hydrate thereof.

12. The method of claim 10 or 11, wherein the level or activity of p53 are increased by about 5-fold, or about 7.5-fold, or about 10 fold, or about 12.5 fold, or about 15 fold.

13. The method of claims 10 to 12, wherein the level or activity of p53 are restored to non-cancerous level or activity in the subject.

14. A method for identifying a subject with a tumor that is likely to respond to treatment with a curaxin or a pharmaceutically acceptable salt or hydrate thereof, comprising evaluating the tumor and/or a blood sample, comprising measuring a presence, absence, level or activity of p53 and/or a NF-kB-responsive marker and/or NF-kB, wherein a reduced level or activity of p53 and/or a NF-kB-resoonsive marker and/or NF-kB relative to a non-tumor state indicates that the subject is likely respond to treatment with a curaxin or a pharmaceutically acceptable salt or hydrate thereof.

15. A method for identifying a subject with a tumor that is likely to respond to treatment with a curaxin or a pharmaceutically acceptable salt or hydrate thereof, comprising evaluating the tumor and/or a blood sample, comprising measuring a ratio of chromatin-bound FACT to soluble FACT, wherein an increased ratio of chromatin-bound FACT to soluble FACT relative to a non-tumor state indicates that the subject is likely respond to treatment with a curaxin or a pharmaceutically acceptable salt or hydrate thereof. 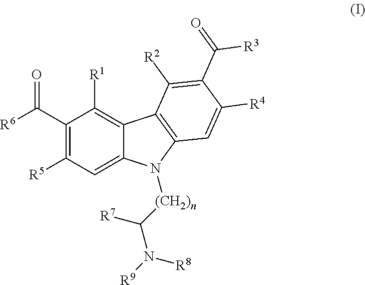
wherein:
each of R¹-R⁹ are independently H, hydroxyl or alkyl and
n is 0, 1, 2, 3, 4, or 5;
or a pharmaceutically acceptable salt or hydrate thereof.

16. The method of claim 14 or 15, comprising, administering an effective amount of the curaxin or a pharmaceutically acceptable salt or hydrate thereof to a subject that is likely to respond to the curaxin or a pharmaceutically acceptable salt or hydrate thereof.

17. The method of claim 14 or 15, wherein the euraxin is a compound of Formula I: 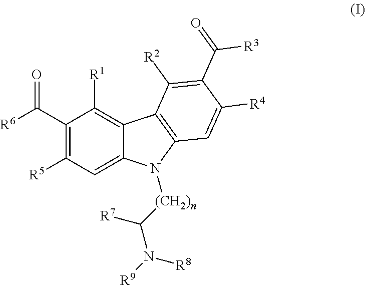
wherein:
each of R¹-R⁹ are independently H, hydroxyl or alkyl and
n is 0, 1, 2, 3, 4, or 5;
or a pharmaceutically acceptable salt or hydrate thereof.

18. The method of claims 14 to 17, wherein the measurement comprises evaluating a presence, absence, level or activity of a protein.

19. The method of any one of claims 14 to 18, wherein the measurement comprises contacting a specimen of the tumor or cells cultured from the tumor with an agent that specifically binds one or more of p53, CXCL10 NF-kB or FACT.

20. The method of claim 19, wherein the agent that specifically binds one or more of p53, CXCL10, NF-kB or FACT is an antibody.

21. The method of any one of claims 14 to 20, wherein the measurement of one or more of p53, CXCL10, NF-kB or FACT comprises one or more of immunohistochemical staining, western blotting, in cell western, immunofiuorescent staining, ELISA, and fluorescent activating cell sorting (FACS).

22. The method of any one of claims 14 to 21, wherein the tumor specimen is a biopsy selected from a frozen tumor tissue specimen, cultured cells, circulating tumor cells, and a formalin-fixed paraffin-embedded tumor tissue specimen.

23. A method for identifying a subject with a tumor that is likely to respond to treatment with an agent that targets or effects p53, comprising administering an effective amount of a curaxin or a pharmaceutically acceptable salt or hydrate thereof and evaluating the tumor and/or a blood sample, comprising measuring a presence, absence, level or activity of p53, wherein an increased level or activity of p53 relative to an untreated state indicates that the subject is likely respond to an agent that targets or effects p53.

24. The method of claim 23, wherein the curaxin is a compound of Formula I: 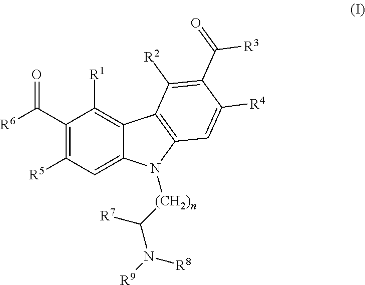
wherein:
each of R′-R⁹ are independently H, hydroxyl or alkyl and
n is 0, 1, 2, 3, 4, or 5;
or a pharmaceutically acceptable salt or hydrate thereof.

25. The method of claims 23 to 24, wherein the measurement comprises evaluating a presence, absence, level or activity of a protein.

26. The method of any one of claims 23 to 25, wherein the measurement comprises contacting a specimen of the tumor or cells cultured from the tumor with an agent that specifically binds p53.

27. The method of clam 26, wherein the agent that specifically binds p53 is an antibody.

28. The method of any one of claims 23 to 27, wherein the measurement of p53 comprises one or more of immunohistochemical staining, western blotting, in cell western, immunofluorescent staining, ELISA, and fluorescent activating cell sorting (FAGS).

29. The method of any one of claims 23 to 28, wherein the tumor specimen is a biopsy selected from a frozen tumor tissue specimen, cultured cells, circulating tumor cells, and a formalin-fixed paraffin-embedded tumor tissue specimen.
